(19) United States
(12) Patent Application Publication
(10) Pub. No.: US 2007/0152208 A1
(43) Pub. Date: Jul. 5, 2007

(54) Optimised method for preparing anellated, polycyclic and polyheterocyclic aromatic compounds

(76) Inventors: Oliver Bartels, Bremen (DE); Olaf Rudiger Hild, Radebeul (DE); Gunter Schnurpfeil, Bemen (DE); Dieter Wohrle, Bremen (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/633,625

(22) Filed: Dec. 5, 2006

(30) Foreign Application Priority Data
Dec. 6, 2005 (DE) 10 2005 058 270.2

(51) Int. Cl. H01L 51/00; C07D 221/18; C07D 335/06

(52) U.S. Cl. 257/40; 257/E51.02; 546/42; 546/61; 549/23

(57) ABSTRACT

The invention relates to a method for producing anellated carbo- or heterocyclic aromatic compounds, which is based on the reduction of the corresponding diketone as starting compound. The reduction agent is thereby used in a strong molar excess, which represents a significant simplification in implementation of the method relative to the methods known from prior art.

The invention relates to a method for producing anellated carbo- or heterocyclic aromatic compounds, which is based on the reduction of the corresponding diketone as starting compound. The reduction agent is thereby used in a strong molar excess, which represents a significant simplification in implementation of the method relative to the methods known from prior art.

Methods from prior art for producing pentacene are known from V. Bruckner, A. Karczag (Wilhelms), K. Körmendy, M. Mezaros and J. Tomasz “Simple Synthesis of Pentacene” Tetrahedron Letters (1959) 5, V. Bruckner, A. Karczag (Wilhelms), K. Körmendy, M. Mezaros and J. Tomasz “Simple and Extensive Synthesis of Pentacene” Acta Chim. Hung. Tomus 22, (1960), 443 and V. Bruckne, and J. Tomasz “Further Simplification of Pentacene Synthesis” Act Chim., Hung. Tomus 28 (1961), 405. The mode of operation described here is based on the reduction of pentacene-6,13-quinone into pentacene, aluminium cyclohexylate being used as reduction agent. With this method implementation, tetrachlorocarbon and mercury(II)chloride are used inter alia as reagents. Both substances are not separated again from the reaction mixture after production of the aluminium cyclohexylate. These compounds, in particular the mercury compounds, must be regarded from the current point of view both with respect to toxicology, disposal and ecology as highly dubious. Furthermore, the method described here involves a very high energy requirement since the implementation of the reaction demands temperatures in the range of 180 to 200° C. The method provides the product with yields of approx. 53%.

Further methods for producing pentacene are known from E. Clar, Fr. John, Ber. 62(1929), 3021; 63(1930), 2967; 64(1931)981, 2194 and W. Bailey, M. Madoff, J. Amer. Chem. Soc. 75(1953), 5603. The methods described here are based however partly on only poorly accessible educts. A further disadvantage of these methods is the significant equipment outlay, the high chemical requirement and the considerable energy costs involved with the method. Thus a reaction via copper at 380° C. is described for example in the case of E. Clar.

Starting herefrom, it was the object of the present invention to provide a method for producing anellated aromatic compounds which enables a simpler method implementation than is known from prior art, and consequently the production costs can be reduced. At the same time, it should concern a method which is less dubious from a toxicological, technical disposal and ecological point of view.

This object is achieved by the method having the features of claim 1, the hereafter produced compound having the features of claim 14 and the use according to claim 16. The further dependent claims reveal advantageous developments.

According to the invention, a method for producing anellated carbo- or heterocyclic aromatic compounds, is provided, which is based on the reduction of a corresponding diketone as starting compound in a one-step synthesis. This diketone has the general formula I

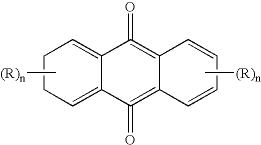

with n respectively independently of each other 1 to 4 and the radicals R respectively independently of each other selected from hydrogen, linear or branched C₁-C₁₈ alkyl, linear or branched C₁-C₁₈ alkoxy, linear or branched C₂-C₈ alkenyl, linear or branched C₂-C₈ alkinyl, C₃-C₈ cycloalkyl, C₅-C₈ aryl, arylalkyl, alkylaryl, arylalkenyl, alkenylaryl, arylalkinyl, alkinylaryl, acyloxy, which can respectively be substituted also by heteroatoms, halogen, radicals R which are respectively adjacent to each other being able to form an aromatic carbo- or heterocyclic ring.

The diketone is converted in lithium aluminium hydride (LiAlH₄) or sodium borohydride with boron trifluoride (NaBH₄.BF₃) as reduction agent, the molar ratio of diketone to reduction agent being in the range of 1:3 to 1:6, preferably of 1:4 to 1:5. The reaction is effected in an organic solvent under protective gas at temperatures in the range of 35 to 120° C. Subsequently, the non-converted reduction agent is deactivated with an acid and the product with the general formula II is isolated.

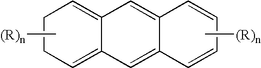

Relative to methods known from prior art, there should be mentioned as essential advantages that the method according to the invention operates at lower temperatures, which leads to a lower energy requirement. Furthermore, no materials are used which are toxicologically or ecologically highly dubious. A further essential advantage is that the implementation of the method can be simplified significantly since, relative to the method according to Vets et al., the number of method steps can be halved. This is associated at the same time with a surprisingly high yield which can be increased up to 80%. Associated with halving the number of method steps there is at the same time also a significantly lower use of organic solvents, likewise the processing of the product is simplified.

It is an important point that the reaction agent is used in a great excess. The diketone in the case of lithium aluminium hydride as reduction agent is probably converted firstly into an aluminium alcoholate compound. This intermediate compound reacts further to form pentacene in a manner not known to date. The supposed mechanism for the reaction is represented in the Figure. Involvement of protons in the actual reaction can be excluded since the product is already present in the reaction mixture before addition of water, which can be detected by the characteristic colouration.

Preferably tetrahydrofuran or ether is used as solvent.

Preferably any non-oxidising acid, in particular hydrochloric acid, is used as acid.

For implementation, the use of protective gas is required, normally argon being used.

Isolation of the product from the reaction solution is generally effected with physical separation methods, in particular the product being suctioned off by means of a frit. It is preferred furthermore that the product is washed and dried subsequent to isolation.

A further essential aspect of the present invention relates to purification of the produced anellated aromatic compounds. Hence, e.g. during the pentacene synthesis, in particular pentacene-6,13-diketone occurs as byproduct, separation of which is required as extensively as possible. According to the invention, the anellated aromatic compound which is produced is suspended at room temperature under protective gas, in particular nitrogen or argon, in xylene or derivatives thereof, in particular o-xylene, the residues of the educts and possibly byproducts dissolving. It is hereby important that the temperature is kept low since anellated aromatic compounds dissolve at higher temperatures in the range of approx. 60 to 120° C. likewise in xylene or derivatives thereof, in particular o-xylene. Since the dissolving process can be observed by a violet colouration, it can be established easily by the person skilled in the art how the temperature should be chosen. In the purification according to the invention, the initially colourless solution becomes coloured yellow by the solution of the educts and byproducts and subsequently is separated. This process is preferably repeated so often until the solution no longer changes colour. After the purification according to the invention, the anellated aromatic compound remains which is preferably dried subsequently. This drying can be effected preferably under vacuum and/or protective gas in order to prevent oxidation of the anellated aromatic compound.

A further improvement with respect to purification can be achieved by recrystalisation under protective gas from hot xylene or derivatives thereof. The use of protective gas and the choice of the correct temperature range are important in the respect that a subsidiary or reverse reaction of the anellated aromatic compound is prevented.

Preferably the method is implemented using a diketone of the general formula III

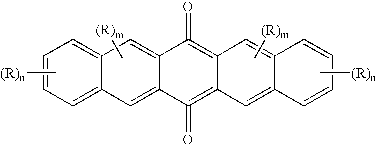

with m=1 to 2 and n=1 to 4 and the radicals R with the above-mentioned meaning. This leads to the current pentacene derivatives.

A further preferred variant relates to the use of a diketone of the general formula IV

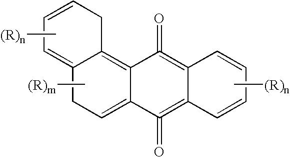

with m=1 or 2 and n=1 to 4 as starting compound. Here also the radicals R have the above-mentioned meaning.

According to the invention, a compound of the general formula II is likewise provided.

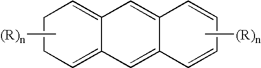

The following meanings apply:

n respectively independently of each other 1 to 4 and the radicals R respectively independently of each other selected from hydrogen, linear or branched C₁-C₁₈ alkyl, linear or branched C₁-C₁₈ alkoxy, linear or branched C₂-C₈ alkenyl, linear or branched C₂-C₈ alkinyl, C₃-C₈ cycloalkyl, C₅-C₈ aryl, arylalkyl, alkylaryl, arylalkenyl, alkenylaryl, arylalkinyl, alkinylaryl, acyloxy, which can respectively be substituted also by heteroatoms, halogen, radicals R which are respectively adjacent to each other being able to form an aromatic carbo- or heterocyclic ring. These compounds are producible according to the previously described method.

A compound of the general formula V is particularly preferred

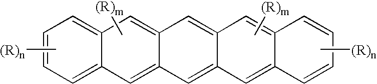

with m=1 to 2 and n=1 to 4. The radicals R thereby have the previously described meaning.

The compounds according to the invention are used as organic semiconductors.

Because of the solubility of pentacene in warm o-xylene, it is possible to produce transistors and circuits with pentacene or derivatives thereof as organic semiconductors. Suitable techniques for this are spincoating, dropcasting, dropcoating, dipcoating, Langmuir-Blodgett. Operating under protective gas is important in order to avoid the oxidation of pentacene.

The method according to the invention is intended to be explained in more detail with reference to the subsequent Figure and the subsequent example without wishing to restrict said method in any way.

The Figure shows a reaction diagram for the implementation of the method according to the invention.

### EXAMPLE 1

1 g (3.3 mmol) pentacene-diketone in 100 ml dry THF is placed in a 250 ml flask. 0.6 g LiAlH₄ are then added at room temperature (RT). Rinsing takes place twice with argon. A reflux cooler is subsequently placed on the flask and the entire system is sealed via a Reitmeyer with an air balloon filled with argon and is refluxed for approx. 60 h in an oil bath at a bath temperature of approx. 110° C. After conclusion of the reduction, the reaction solution is coloured dark green with a yellow tinge. It is then left to cool to approximately RT without removing the bath in order to assist formation of larger pentacene crystals, which makes suctioning off easier. Then 30 ml HCI (6 mol/l) are added in drops with ice cooling in order to deactivate any not yet consumed lithium aluminium hydride and in order to dissolve any lithium and aluminium hydroxide produced and to remove them by suctioning off. Agitation then takes place for another 30 min. at RT and suctioning off via a P4 frit. Then the reaction product is washed with 50 ml 6M HCI in smaller batches, 50 ml water in smaller batches, approx. 30 ml acetone and 10 ml ether. After drying for 20 minutes, 726 mg (2.6 mmol=80% of the theoretical) of a violet substance is obtained. Reducing the reaction time to 40 h leads to a yield of approx. 70% of the theoretical. Increasing the reaction time to approx. 96 h does not lead to an improvement in the yield. The yields of the reaction are in the range of 1 to 10 g educt, independently of the quantity of educt.

Characterisation:

Mass spectrometry: the substance is pure according to mass spectrometry: Base peak=molecule ion in El: 278. Further signal at 252 and 139 (M²⁺). The mass spectrum hence corresponds to the data of the literature.

IR [cm⁻¹]: 3044 (w, aryl-H valency), 1625 (w, carbonyl: this signal is caused by the smallest residues of educts and by air oxidation. In view of the normally very intensive CO oscillation in the IR and in connection with the results of the mass spectrometry analysis, this signal is negligible. Even in twice sublimated pentacene, this signal can still be observed), 1296 (w), 957 (w), 907 (s, aryl-H deformation), 732 (s, aryl-H deformation), 468 (m).

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding German application No. DE 10 2005 058 270.2, filed Dec. 6, 2005, is incorporated by reference herein.

The preceding examples can be repeated with similar success by substituting the generically or specifically described reactants and/or operating conditions of this invention for those used in the preceding examples. From the foregoing description, one skilled in the art can easily ascertain the essential characteristics of this invention and, without departing from the spirit and scope thereof, can make various changes and modifications of the invention to adapt it to various usages and conditions.

1. Method for producing anellated carbo- or heterocyclic aromatic compounds in which, in a single-step synthesis, a diketone of the general formula I 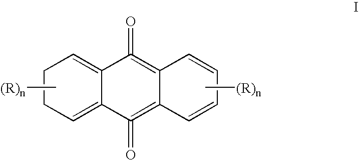
with n respectively independently of each other 1 to 4 and the radicals R respectively independently of each other selected from hydrogen, linear or branched C₁-C₁₈ alkyl, linear or branched C₁-C₁₈ alkoxy, linear or branched C₂-C₈ alkenyl, linear or branched C₂-C₈ alkinyl, C₃-C₈ cycloalkyl, C₅-C₈ aryl, arylalkyl, alkylaryl, arylalkenyl, alkenylaryl, arylalkinyl, alkinylaryl, acyloxy, which can respectively be substituted also by heteroatoms, halogen, radicals R which are respectively adjacent to each other being able to form an aromatic carbo- or heterocyclic ring
with lithium aluminium hydride (LiAlH₄) or sodium borohydride with boron trifluoride (NaBH₄.BF₃) as reduction agent, the molar ratio of diketone to reduction agent being in the range of 1:3 to 1:6, is used, is converted in an organic solvent under protective gas at temperatures in the range of 35 to 120° C., the non-converted reacted reduction agent is deactivated with an acid and the product with the general formula II 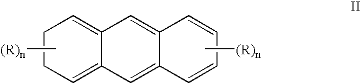
is isolated, the radicals having the above-indicated meaning.

2. Method according to claim 1,
characterised in that the heteroatoms are selected from the group comprising oxygen, sulphur and nitrogen.

3. Method according to claim 1, characterised in that tetrahydrofuran or ether is used as solvent.

4. Method according to claim 1, characterised in that hydrochloric acid is used as acid.

5. Method according to claim 1, characterised in that argon is used as protective gas.

6. Method according to claim 1, characterised in that the isolation is effected by means of physical separation methods, in particular by suctioning off.

7. Method according to claim 1, characterised in that the product is dissolved at least once in xylene or derivatives thereof at temperatures of 15 to 30° in order to separate byproducts and purify the product under protective gas and it is isolated from the dissolved byproducts.

8. Method according to claim 1, characterised in that the product is recrystalised in xylene or derivatives thereof at temperatures of 60 to 150° C. in order to separate byproducts and to purify the product under protective gas.

9. Method according to claim 1, characterised in that the product is washed and dried subsequent to the isolation.

10. Method according to claim 1, characterised in that diketone of the general formula III is used 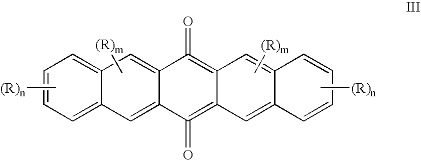
with m=1 to 2 and n=1 to 4 and the radicals R have the meaning mentioned in claim 1.

11. Method according to claim 1, characterised in that the radicals R are respectively hydrogen.

12. Method according to claim 1, characterised in that a diketone of the general formula IV is used 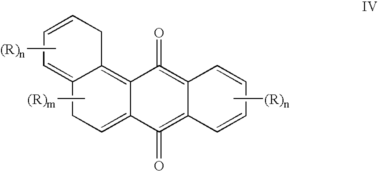
with m=1 to 2 and n=1 to 4 and the radicals R can have the above-mentioned meaning.

13. Method according to claim 1, characterised in that the radicals R are respectively hydrogen.

14. Compound of the general formula II 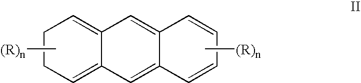
with n respectively independently of each other 1 to 4 and the radicals R respectively independently of each other selected from hydrogen, linear or branched C₁-C₁₈ alkyl, linear or branched C₁-C₁₈ alkoxy, linear or branched C₂-C₈ alkenyl, linear or branched C₂-C₈ alkinyl, C₃-C₈ cycloalkyl, C₅-C₈ aryl, arylalkyl, alkylaryl, arylalkenyl, alkenylaryl, arylalkinyl, alkinylaryl, acyloxy, which can respectively be substituted also by heteroatoms, halogen, radicals R which are respectively adjacent to each other being able to form an aromatic carbo- or heterocyclic ring,
producible according to the method according to claim 1.

15. Compound according to claim 14, characterised by the general formula V 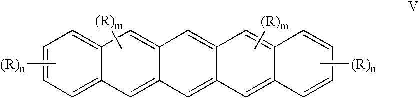
with m=1 to 2 and n=1 to 4 and the radicals R respectively independently of each other selected from hydrogen, linear or branched C₁-C₁₈ alkyl, linear or branched C₁-C₁₈ alkoxy, linear or branched C₂-C₈ alkenyl, linear or branched C₂-C₈ alkinyl, C₃-C₈ cycloalkyl, C₅-C₈ aryl, arylalkyl, alkylaryl, arylalkenyl, alkenylaryl, arylalkinyl, alkinylaryl, acyloxy, which can respectively be substituted also by heteroatoms, halogen, radicals R which are respectively adjacent to each other being able to form an aromatic carbo- or heterocyclic ring.

16. Use of the compound according to claim 14 as organic semiconductor, in particular in transistors or circuits.
